(19) United States
(12) Patent Application Publication
(10) Pub. No.: US 2025/0032499 A1
(43) Pub. Date: Jan. 30, 2025

(54) METHODS OF TREATING PERIPHERAL T-CELL LYMPHOMA

(71) Applicant: Secura Bio, Inc., Las Vegas, NV (US)

(72) Inventors: Debra LITWAK, Las Vegas, NV (US); David COHAN, Las Vegas, NV (US)

(21) Appl. No.: 18/711,709

(22) PCT Filed: Nov. 22, 2022
(86) PCT No.: PCT/US2022/050712

§ 371 Date: May 20, 2024

Related U.S. Application Data
(60) Provisional application No. 63/281,998, filed on Nov. 22, 2021.

(51) Int. Cl. A61K 31/52

(52) U.S. Cl. CPC A61K 31/52

(57) ABSTRACT

The present disclosure provides methods for treatment peripheral T-cell lymphoma with duvelisib.

### CROSS-REFERENCE TO RELATED APPLICATIONS

This application claims the benefit of and priority to U.S. Provisional Application No. 63/281,998, filed Nov. 22, 2021, which is incorporated herein by reference in its entirety.

### BACKGROUND

Lymphoma is the most common blood cancer. The two main forms of lymphoma are Hodgkin lymphoma and non-Hodgkin lymphoma (NHL). Lymphoma occurs when cells of the immune system called lymphocytes, a type of white blood cell, grow and multiply uncontrollably. Cancerous lymphocytes can travel to many parts of the body, including the lymph nodes, spleen, bone marrow, blood, or other organs, and form a mass called a tumor. The body has two main types of lymphocytes that can develop into lymphomas: B-lymphocytes (B-cells) and T-lymphocytes (T-cells). T-cell lymphomas account for approximately 15 percent of all NHLs in the United States. Most T-cell lymphomas can be classified into two broad categories: aggressive (fast-growing) or indolent (slow-growing).

Peripheral T-cell lymphoma (PTCL) is a group of rare and usually aggressive (fast-growing) NHLs that develop from mature T-cells. PTCLs are sub-classified into various subtypes. Most of these subtypes are very rare; the three most common subtypes of PTCL, peripheral T-cell lymphoma not otherwise specified (PTCL-NOS), anaplastic large-cell lymphoma (ALCL), and angioimmunoblastic T-cell lymphoma (AITL), and these account for approximately 70 percent of all PTCLs in the United States.

PTCL is generally associated with poor prognosis, and survival outcome for patients with PTCL who relapse or progress after first-line treatment is very poor. Current FDA approved therapies for relapsed or refractory (R/R) PTCL have modest overall response rates (ORR) of <30%, and the median overall survival (OS) of less than 6 months. Thus, new methods of treating PTCL are required to overcome this urgent unmet treatment need. The present disclosure addresses this need.

### SUMMARY OF THE DISCLOSURE

In embodiments, the present disclosure is a method of treating peripheral T-cell lymphoma (PTCL) in a subject in need thereof, comprising orally administering to the subject for three or more treatment cycles duvelisib having the following structure:

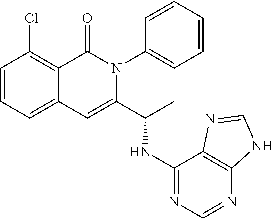 [SMILES: C[C@H](Nc1ncnc2[nH]cnc12)c1cc2cccc(Cl)c2c(=O)n1-c1ccccc1]

or a pharmaceutically acceptable salt or hydrate thereof;
wherein about 75 mg of duvelisib is administered twice daily for first two treatment cycles; and
wherein about 25 mg of duvelisib is administered twice daily for at least the third treatment cycle.

In embodiments, duvelisib is a hydrate.

In embodiments, each treatment cycle has a duration of about 28 days.

In embodiments, 25 mg of duvelisib is administered twice daily in one or more treatment cycles following the third treatment cycle. In embodiments, 25 mg of duvelisib is administered twice daily in each treatment cycle following the third treatment cycle.

In embodiments, the method comprises at least a fourth, fifth, six, seventh, eighth, ninth, tenth, eleventh, and twelfth treatment cycle, and the method comprises administering 25 mg of duvelisib twice daily in one or more of the fourth, fifth, six, seventh, eighth, ninth, tenth, eleventh, and twelfth treatment cycles. In embodiments, the method comprises at least a fourth, fifth, six, seventh, eighth, ninth, tenth, eleventh, and twelfth treatment cycles, and the method comprises administering 25 mg of duvelisib twice daily in two, three, four, five, six, seven, eight, nine, or ten of the fourth, fifth, six, seventh, eighth, ninth, tenth, eleventh, and twelfth treatment cycles.

In embodiments, the PTCL is peripheral T-cell lymphoma, not otherwise specified (PTCL-NOS), anaplastic large cell lymphoma (ALCL), natural-killer/T-cell lymphoma (NKTL), or angioimmunoblastic T-cell lymphoma (AITL). In embodiments, the PTCL is PTCL-NOS. In embodiments, the PTCL is ALCL. In embodiments, the PTCL is AITL. In embodiments, the PTCL is NKTL.

In embodiments, the lymphoma is relapsed or refractory. In embodiments, the subject received at least 2 cycles of one prior regimen for PTCL and:
a. failed to achieve at least a partial response after 2 or more cycles;
b. failed to achieve a complete response after 6 or more cycles; and/or
c. progressed after an initial response.

In embodiments, the method comprises administering the subject prophylaxis treatment to pneumocystis jirovecii pneumonia (PM), herpes simplex virus (HSV), varicella zoster virus (VZV) prophylaxis, or combinations thereof.

In embodiments, treatment increases the overall response rates (ORR) compared to either (i) the median ORR of patients administered standard treatment for PTCL or (ii) the ORR of an otherwise similar patient with PTCL that is not treated with duvelisib.

In embodiments, treatment increases progression free survival (PFS) compared to either (i) the median PFS of patients administered standard treatment for PTCL or (ii) the PFS of an otherwise similar patient with PTCL that is not treated with duvelisib.

In embodiments, treatment increases overall survival (OS) compared to either (i) the median OS of patients administered standard treatment for PTCL or (ii) the OS of an otherwise similar patient with PTCL that is not treated with duvelisib.

In embodiments, treatment increases duration of response (DOR) to either (i) the median DOR of patients administered standard treatment for PTCL or (ii) the DOR of an otherwise similar patient with PTCL that is not treated with duvelisib.

In embodiments, treatment increases the disease control rate (DCR) to either (i) the median DCR of patients administered standard treatment for PTCL or (ii) the DCR of an otherwise similar patient with PTCL that is not treated with duvelisib.

### DETAILED DESCRIPTION OF THE INVENTION

### Definitions

Any term or expression not expressly defined herein shall have its commonly accepted definition understood by those skilled in the art. To the extent that the following description is of a specific embodiment or a particular use of the invention, it is intended to be illustrative only, and not limiting of the claimed invention. The following description is intended to cover all alternatives, modifications and equivalents that are included in the spirit and scope of the invention, as defined in the appended claims.

As used in the specification and claims, the singular form “a”, “an” and “the” includes plural references unless the context clearly dictates otherwise.

As used herein, and unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The terms “antagonist” and “inhibitor” are used interchangeably, and they refer to a compound or agent having the ability to reduce or inhibit a biological function of a target protein or polypeptide, such as by reducing or inhibiting the activity or expression of the target protein or polypeptide. Accordingly, the terms “antagonist” and “inhibitor” are defined in the context of the biological role of the target protein or polypeptide. An inhibitor need not completely abrogate the biological function of a target protein or polypeptide, and in some embodiments reduces the activity by at least 50%, 60%, 70%, 80%, 90%, 95%, or 99%. While some antagonists herein specifically interact with (e.g., bind to) the target, compounds that inhibit a biological activity of the target protein or polypeptide by interacting with other members of the signal transduction pathway of which the target protein or polypeptide are also specifically included within this definition. Non-limiting examples of biological activity inhibited by an antagonist include those associated with the development, growth, or spread of a tumor, or an undesired immune response as manifested in autoimmune disease.

The term “therapeutically effective amount” refers to that amount of a compound or pharmaceutical composition described herein that is sufficient to effect the intended application including, but not limited to, disease treatment, as illustrated below. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific dose will vary depending on, for example, the particular compounds chosen, the dosing regimen to be followed, whether it is administered in combination with other agents, timing of administration, the tissue to which it is administered, and the physical delivery system in which it is carried.

As used herein, a daily dosage can be achieved by a single administration of the targeted dosage amount or multiple administrations of smaller dosage amount(s). For example, a 150 mg daily dosage can be achieved by a single administration of 150 mg of the therapeutic agent per day, two administrations of 75 mg of the therapeutic agent per day, or three administrations of 50 mg of the therapeutic agent per day, or the like.

As used herein, “treat” or “treating” means one or more of relieving, alleviating, delaying, reducing, reversing, improving, or managing at least one symptom of a condition in a subject. The term “treating” may also mean one or more of arresting, delaying the onset (i.e., the period prior to clinical manifestation of the condition) or reducing the risk of developing or worsening a condition.

As used herein, the term “duvelisib” refers to the compound having the chemical structure:

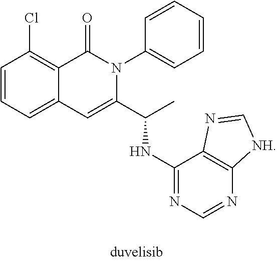 [SMILES: C[C@H](Nc1ncnc2[nH]cnc12)c1cc2cccc(Cl)c2c(=O)n1-c1ccccc1]

The chemical name for duvelisib is (S)-3-(1-((9H-purin-6-yl)amino)ethyl)-8-chloro-2-phenylisoquinolin-1(2H)-one.

In certain embodiments, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J Pharmaceutical Sciences (1977) 66: 1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts may be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

Pharmaceutically acceptable salts derived from appropriate bases include alkali metal, alkaline earth metal, ammonium and N(C₁₋₄ alkyl)₄ salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate. Organic bases from which salts may be derived include, for example, primary, secondary, and tertiary amines, substituted amines including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

In certain embodiments, the pharmaceutically acceptable form is a solvate (e.g., a hydrate). As used herein, the term “solvate” refers to compounds that further include a stoichiometric or non-stoichiometric amount of solvent bound by non-covalent intermolecular forces. The solvate may be of a disclosed compound or a pharmaceutically acceptable salt thereof. Where the solvent is water, the solvate is a “hydrate”. Pharmaceutically acceptable solvates and hydrates are complexes that, for example, can include 1 to about 100, or 1 to about 10, or one to about 2, about 3 or about 4, solvent or water molecules. It will be understood that the term “compound” as used herein encompasses the compound and solvates of the compound, as well as mixtures thereof.

As used herein, and unless otherwise specified, “polymorph” may be used herein to describe a crystalline material, e.g., a crystalline form. In certain embodiments, “polymorph” as used herein are also meant to include all crystalline and amorphous forms of a compound or a salt thereof, including, for example, crystalline forms, polymorphs, pseudopolymorphs, solvates, hydrates, co-crystals, unsolvated polymorphs (including anhydrates), conformational polymorphs, tautomeric forms, disordered crystalline forms, and amorphous forms, as well as mixtures thereof, unless a particular crystalline or amorphous form is referred to. Compounds of the present disclosure include crystalline and amorphous forms of those compounds, including, for example, crystalline forms, polymorphs, pseudopolymorphs, solvates, hydrates, co-crystals, unsolvated polymorphs (including anhydrates), conformational polymorphs, tautomeric forms, disordered crystalline forms, and amorphous forms of the compounds or a salt thereof, as well as mixtures thereof.

All references to a drug herein, like a PI3K inhibitor such as duvelisib, include pharmaceutically acceptable salts, solvates, hydrates, and polymorphs of the said drug. Examples of salts, solvates, hydrates, and polymorphs of duvelisib are described in WO 2012/097000, which are incorporated herein by reference for all purposes.

### Methods of the Present Disclosure

The present disclosure relates to safe and effective methods of treating peripheral T-cell lymphoma (PTCL). In embodiments, the disclosure provides a method of treating PTCL in a subject in need thereof, comprising administering to the subject a therapeutically effective amount of a PI3K inhibitor described herein in three or more treatment cycles, wherein the daily dose of the PI3K inhibitor in the first two treatment cycles is higher than a total daily dose of the PI3K inhibitor in the third treatment cycle and optionally one or more subsequent treatment cycles. In embodiments, the PI3K is duvelisib having the following structure:

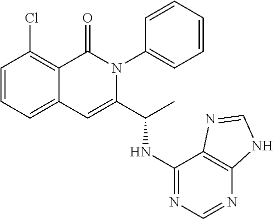 [SMILES: C[C@H](Nc1ncnc2[nH]cnc12)c1cc2cccc(Cl)c2c(=O)n1-c1ccccc1]

or a pharmaceutically acceptable salt or hydrate thereof. In embodiments, about 75 mg of duvelisib is administered twice daily for first two treatment cycles. In embodiments, about 25 mg of duvelisib is administered twice daily for at least the third treatment cycle.

Prior to the present disclosure, Applicant ran a clinical trial using duvelisib to treat PTCL. The trial included two cohorts. In cohort 1, subjects were orally administered duvelisib at a starting dose of 25 mg twice daily (BID), with potential escalation on a per-patient basis to 50 mg BID and then 75 mg BID, based on the patient's response to and tolerance of therapy. Doses were administered in 28-day cycles. In cohort 2, subjects were orally administered 75 mg duvelisib BID, in 28-day cycles. The overall response rate (ORR) in the 75 mg BID cohort was 54% ORR. In the 25 mg BID cohort, the ORR was significantly lower, at 35%, and three times as many patients experienced disease progression compared to patients receiving 75 mg BID. While the ORR was significantly higher in the 75 mg BID cohort, patients experienced significant adverse events, including infectious colitis and sepsis, whereas patients in the 25 mg BID cohort did not experience infectious colitis and sepsis. Surprisingly, Applicant discovered that most treatment emergent adverse events were manageable with dose reductions, and such dose reductions did not result in a corresponding reduction in efficacy.

Thus, Applicant identified a dosing regimen in which subjects initially receive a higher dose, and then the dose is lowered. Without being limited to a particular theory, it was unexpectedly found that treatment with a high dosage followed by lower dosage cycles maximized rapid tumor control, while also maintaining long-term control and mitigating toxicities. More specifically, the initial higher doses address aggressive stages of PTCL, and while the lower dose minimizes side effects that otherwise would occur if administration of the higher dose was continued. Surprisingly, the lower dose maintains efficacy and does not exhibit a significant drop in efficacy compared to the higher dose. In embodiments, the higher dose is 75 mg BID (150 mg total daily dose). In embodiments, the lower dose is 25 mg BID (50 mg total daily dose).

In embodiments, the PTCL is peripheral T-cell lymphoma not otherwise specified (PTCL-NOS), anaplastic large cell lymphoma (ALCL), natural-killer/T-cell lymphoma (NKTL), or angioimmunoblastic T-cell lymphoma (AITL). In embodiments, the PTCL is PTCL-NOS. In embodiments, the PTCL is ALCL. In embodiments, the PTCL is AITL. In embodiments, the PTCL is NKTL.

In embodiments, PTCL is relapsed or refractory. In embodiments, the PTCL is relapsed. In embodiments, the PTCL is refractory. In embodiments, the subject received at least two cycles of one prior regimen for PTCL and one of the following:
a. failed to achieve at least a partial response after 2 or more cycles;
b. failed to achieve a complete response after 6 or more cycles; and/or
c. progressed after an initial response.

In embodiments, the prior regimen for PTCL may include, but is not limited to, cyclophosphamide, doxorubicin, vincristine, prednisone, etoposide, prednisone, and combinations thereof. In embodiments, the prior regimen for PTCL may include a combination chemotherapy regimen, such as CHOP (cyclophosphamide, doxorubicin, vincristine, and prednisone) or CHOEP (etoposide, vincristine, doxorubicin, cyclophosphamide, and prednisone).

In embodiments, the methods comprising administering prophylaxis treatment for pneumocystis jirovecii pneumonia (PJP), herpes simplex virus (HSV), varicella zoster virus (VZV) prophylaxis, or combinations thereof. In embodiments, the subject is administered prophylaxis treatment for pneumocystis jirovecii pneumonia (PJP). In embodiments, the subject is administered prophylaxis treatment for herpes simplex virus (HSV). In embodiments, the subject is administered prophylaxis treatment for varicella zoster virus (VZV) prophylaxis. In embodiments, prophylaxis treatment for PIP occurs during treatment with duvelisib and continues until the absolute CD4+ T cell count is greater than 200 cells/μL.

### PI3K Inhibitors

In embodiments, the PI3K inhibitor has a structure according to Formula I:

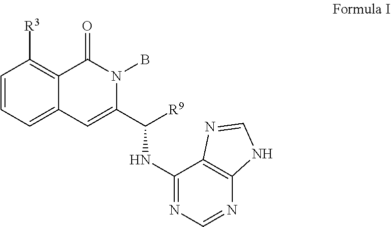

or a pharmaceutically acceptable salt thereof,
wherein B is a moiety of Formula II:

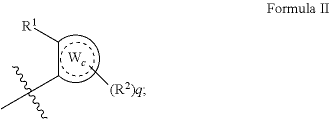

W_{c} is aryl, heteroaryl, heterocycloalkyl, or cycloalkyl;
q is an integer of 0 or 1;
R¹ is hydrogen, alkyl, or halo;
R² is alkyl or halo;
R³ is halo; and
R⁹ is alkyl, or heterocycloalkyl;

In embodiments, Wₑ is aryl. In embodiments, q is 0. In embodiments, R¹ is hydrogen. R³ is —Cl. In embodiments, R⁹ is —CH₃.

In embodiments, the PI3K inhibitor is described in WO 2011/008302, which is herein incorporated by reference in its entirety, for all purposes.

In embodiments, the PI3K inhibitor is duvelisib, AEZS-136, alpelisib, buparlisib, CAL263, copanlisib, CUDC-907, dactolisib, GNE-477, GSK1059615, IC87114, idelalisib, INK1117, IPI-549, leniolisib, ME-401, palomid-529, parsaclisib, paxalisib, perifosine, PI-103, pictilisib, PWT33597, PX-866, RP6503, RP6530, SF1126, taselisib, TG100-115, umbralisib, voxtalisib, XL147, zandelisib, or ZSTK474, or pharmaceutically acceptable salts or hydrates thereof. In embodiments, the PI3K inhibitor is duvelisib, idelalisib, copanlisib, parsaclisib, paxalisib, umbralisib, or zandelisib, or pharmaceutically acceptable salts or hydrates thereof. The structure of these compounds is provided below:

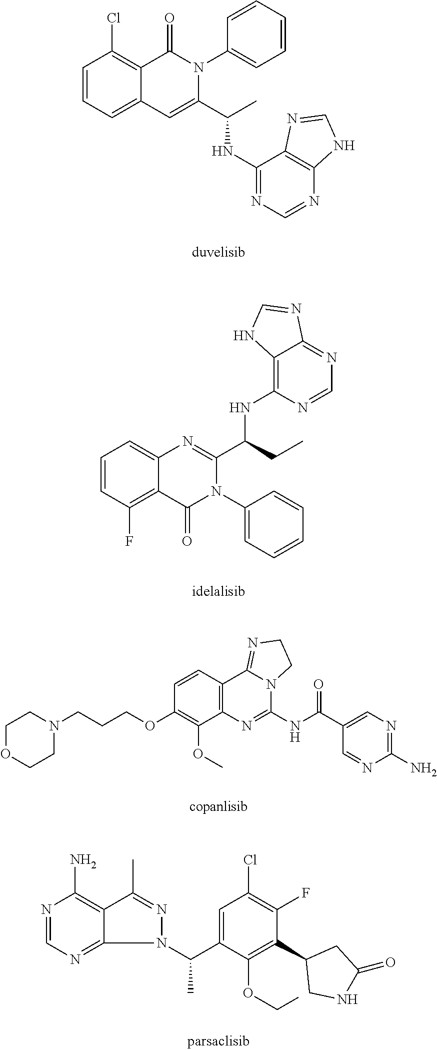 [SMILES: C[C@H](Nc1ncnc2[nH]cnc12)c1cc2cccc(Cl)c2c(=O)n1-c1ccccc1 ; CC[C@H](Nc1ncnc2nc[nH]c12)c1nc2cccc(F)c2c(=O)n1-c1ccccc1 ; COc1c(OCCCN2CCOCC2)ccc2c1N=C(NC(=O)c1cnc(N)nc1)N1CCN=C21 ; CCOc1c([C@H](C)n2nc(C)c3c(N)ncnc32)cc(Cl)c(F)c1[C@@H]1CNC(=O)C1]

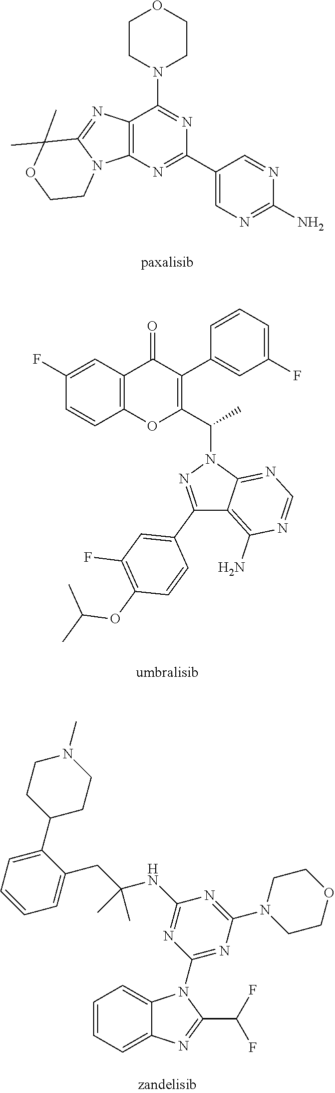 [SMILES: CC1(C)OCCn2c1nc1c(N3CCOCC3)nc(-c3cnc(N)nc3)nc12 ; CC(C)Oc1ccc(-c2nn([C@@H](C)c3oc4ccc(F)cc4c(=O)c3-c3cccc(F)c3)c3ncnc(N)c23)cc1F ; CN1CCC(c2ccccc2CC(C)(C)Nc2nc(N3CCOCC3)nc(-n3c(C(F)F)nc4ccccc43)n2)CC1]

In embodiments, the PI3K inhibitor is duvelisib or a pharmaceutically acceptable salt or hydrate thereof. In embodiments, duvelisib is a hydrate.

### Dosing and Administration

In embodiments, a higher total daily dose of the PI3K inhibitor (e.g., duvelisib) is administered for a period of time (e.g., two or more treatment cycles), followed by administering a lower total daily dose of the PI3K inhibitor (e.g., duvelisib) for a period of time (e.g., one or more of the subsequent treatment cycles). In embodiments, the higher dose can be administered for 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, or more. In embodiments, following administration of the higher dose, the lower dose can be administered for 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 13 months, 14 months, 15 months, 16 months, 17 months, 18 months, 19 months, 20 months, 21 months, 22 months, 23 months, 24 months or more.

In embodiments, the methods of the disclosure comprise 1, 2, 3, 4, 5, 6, 7, 8, 9, 10, 11, 12, 13, 14, 15, 16, 1,7 18, 19, 20, 21, 22, 23, 24, 25, 26, 27, 2,8 29, 30, 31, 32, 33, 34, 35, 36, 37, 38, 39, 40, 41, 42, 43, 44, 45,46, 47, 48, 49, 50 or more treatment cycles, including all values and ranges in between. In embodiments, the total daily dose of the PI3K inhibitor (e.g., duvelisib) in the first round of treatment cycles is higher than a total daily dose of the PI3K (e.g., duvelisib) inhibitor in one or more subsequent treatment cycles. In embodiments, the first round of treatment cycles may comprise 1, 2, 3, 4, or 5 cycles. In embodiments, the first round of treatment cycles comprises 2 treatment cycles.

In embodiments, the total daily dose of the PI3K inhibitor (e.g., duvelisib) in each of the first and second treatment cycle is higher than the total daily dose of the PI3K inhibitor (e.g., duvelisib) in one or more subsequent treatment cycles. In embodiments, the total daily dose of the PI3K inhibitor (e.g., duvelisib) in each of the first and second treatment cycle is higher than the total daily dose of the PI3K inhibitor (e.g., duvelisib) in the third treatment cycle. In embodiments, the total daily dose of the PI3K inhibitor (e.g., duvelisib) in each of the first and second treatment cycle is higher than the total daily dose of the PI3K inhibitor in the third treatment cycle, fourth treatment cycle, fifth treatment cycle, sixth treatment cycle, and so on. In embodiments, the total daily dose in first and second treatment cycles are the same or different. In embodiments, the total daily dose in first and second treatment cycles are the same.

In embodiments, the total daily dose of the PI3K inhibitor (e.g., duvelisib) administered in each of the first and second treatment cycles is about 150% to about 500% greater than the total daily dose administered in the third cycle (and optionally one or more subsequent treatment cycle). For example, the first round of treatment cycles can be about 150%, about 175%, about 200%, about 225%, about 250%, about 275%, about 300%, about 325%, about 350%, about 375%, about 400%, about 425%, about 450%, about 475%, or about 500% greater the total daily dose administered in the third treatment cycle (and optionally one or more subsequent treatment cycle). In embodiments, the PI3K inhibitor is duvelisib, and the total daily dose of duvelisib administered in each of the first and second treatment cycles is about 300% greater than the total daily dose of duvelisib administered in the third treatment cycle (and optionally one or more subsequent treatment cycle).

In embodiments, the total daily dose of the PI3K inhibitor (e.g., duvelisib) administered in each of the first and second treatment cycles is at least about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg greater than the total daily dose administered in the third cycle (and optionally one or more subsequent treatment cycle). In embodiments, the PI3K inhibitor is duvelisib, and the total daily dose of duvelisib administered in each of the first and second treatment cycles is 50 mg greater than the total daily dose of duvelisib administered in the third treatment cycle (and optionally one or more subsequent treatment cycle).

In embodiments, the total daily dose of the PI3K inhibitor (e.g., duvelisib) administered in each of the first and second cycles is about 100 mg to about 200 mg, e.g., about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg, including all values and ranges in between. In embodiments, the PI3K inhibitor is duvelisib, and the total daily dose of duvelisib administered in each of the first and second treatment cycles is about 150 mg.

In embodiments, the total daily dose of the PI3K inhibitor (e.g., duvelisib) administered in the third treatment cycle (and optionally one or more subsequent treatment cycle) is about 40 mg to about 60 mg, e.g., about 40 mg, about 45 mg, about 50 mg, about 55 mg, or about 60 mg. In embodiments, the PI3K inhibitor is duvelisib, and the total daily dose of duvelisib administered in the third treatment cycle (and optionally one or more subsequent treatment cycle) is about 50 mg.

In embodiments, the PI3K inhibitor is administered once or twice daily. In embodiments, the PI3K inhibitor is administered once daily. In embodiments, the PI3K inhibitor is administered twice daily. In embodiments, the PI3K inhibitor is duvelisib. In embodiments, about 75 mg of duvelisib is administered twice daily during each of the first and second treatment cycles. In embodiments, about 25 mg of duvelisib is administered twice daily during the third treatment cycle. In embodiments, about 25 mg of duvelisib is administered twice daily during the third, fourth, fifth, sixth, seventh, eighth, nineth, tenth, eleventh, twelfth, or more treatment cycle. In embodiments, about 25 mg of the PI3K inhibitor is administered twice daily during the third treatment cycle and throughout the remainder of treatment.

In embodiments a treatment cycle has a duration of about 7 days to about 31 days. For example, in embodiments, a treatment cycle has a duration of about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days, including all ranges in between. In embodiments, a treatment cycle has a duration of about 28 days.

In embodiments, the method of the disclosure comprises a rest period between cycles. A “rest period” as described herein is a period of time wherein the subject does not receive a PI3K inhibitor of the disclosure. In embodiments, a rest period has a duration of about 1 to about 31 days. For example, a rest period has about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days, including all values and ranges in between. In embodiments, a rest period follows one or more of the cycles. In embodiments, each rest period follows the first cycle, the second cycle, the third cycle, the fourth cycle, the fifth cycle, the sixth cycle, the seventh cycle, and so on. In embodiments, each the period of time in each rest period may be the same or different. In embodiments, the methods do not comprise a rest period between cycles.

### Clinical Endpoints

In embodiments, the treatment increases the overall response rates (ORR) compared to either (i) the median ORR of patients administered standard treatment for PTCL or (ii) the ORR of an otherwise similar patient with PTCL that is not treated with the duvelisib (e.g., a control patient). As defined herein, ORR is the proportion of patients in which a complete response (CR) or partial response (PR) is observed. In embodiments, treating according to the methods of the disclosure results in an increase in ORR as compared to the median value in ORR at the time of this disclosure for PTCL. In embodiments, the standard treatment for PTCL is chemotherapy, for example BV-CHP (combination of brentuximab vedotin, cyclophosphamide, doxorubicin, and prednisolone), CHOEP (cyclophosphamide, doxorubicin, vincristine, etoposide, prednisone combination), CHOP (cyclophosphamide, doxorubicin, vincristine, prednisone combination), EPOCH (etoposide, prednisone, vincristine, cyclophosphamide, doxorubicin), and R-CHOP (rituximab, cyclophosphamide, doxorubicin, vincristine, prednisone combination). In embodiments, the standard treatment for PTCL is chemotherapy or stem cell transplantation (SCT).

In embodiments, treating according to the methods of the disclosure results in an increase in the ORR of about 10%, about 20%, about 30%, about 40%, about 50%, about 60%, about 70%, about 80%, about 90%, about 100%, about 150%, about 200%, about 250%, about 300%, about 350%, about 400%, about 450%, about 500%, about 550%, about 600%, about 650%, about 700%, about 750%, about 800%, about 850%, about 900%, about 950%, or about 1000% or more, including all values and ranges in between, compared to the (i) the median ORR of patients administered standard treatment for PTCL or (ii) the ORR of an otherwise similar patient with PTCL that is not treated with the duvelisib.

In embodiments, treating according to the methods of the disclosure results in an ORR of about 40%, about 45%, about 50%, about 55%, about 60%, about 65%, about 70%, about 75%, about 80%, about 85%, about 90%, about 95%, about 99%, or about 100%, including all values and ranges in between.

In embodiments, the treatment increases progression free survival (PFS) compared to either (i) the median PFS of patients administered standard treatment for PTCL or (ii) the PFS of an otherwise similar patient with PTCL that is not treated with duvelisib (e.g., a control patient). As used herein, PFS is the length of time between initiation of treatment according to the methods of the disclosure and the date of documented progressive disease or death from any cause.

In embodiments, treating according to the methods of the disclosure results in an increase in the PFS of about 1 week, or about 2 weeks, or about 3 weeks, or about 4 weeks, or about 5 weeks, or about 6 weeks, or about 7 weeks, or about 8 weeks, or about 9 weeks, or about 10 weeks, or about 1 month, or about 2 months, or about 3 months, or about 4 months, or about 5 months, or about 6 months, or about 7 months, or about 8 months, or about 9 months, or about 10 months, or about 11 months, or more, or about 12 months, or about 13 months, or about 14 months, or 15 months, or about 16 months, or about 17 months, or about 18 months, or about 19 months, or about 20 months, or about 21 months, or about 22 months, or about 23 months, or about 24 months, or about 3 years, or about 4 years, or about 5 years, or about 6 years, or about 7 years, or about 8 years, or about 9 years, or about 10 years, including all values and ranges in between, compared to the (i) the median PFS of patients administered standard treatment for PTCL or (ii) the PFS of an otherwise similar patient with PTCL that is not treated with duvelisib.

In embodiments, treating according to the methods of the disclosure results in an increase in the PFS of about 10%, about 20%, about 30%, about 40%, about 50%, about 60%, about 70%, about 80%, about 90%, about 100%, about 150%, about 200%, about 250%, about 300%, about 350%, about 400%, about 450%, about 500%, about 550%, about 600%, about 650%, about 700%, about 750%, about 800%, about 850%, about 900%, about 950%, or about 1000% or more, including all values and ranges in between, compared to the (i) the median PFS of patients administered standard treatment for PTCL or (ii) the PFS of an otherwise similar patient with PTCL that is not treated with duvelisib.

In embodiments, treating according to the methods of the disclosure results in a PFS of about 12 months, about 13 months, or about 14 months, or 15 months, or about 16 months, or about 17 months, or about 18 months, or about 19 months, or about 20 months, or about 21 months, or about 22 months, or about 23 months, or about 24 months, or about 3 years, or about 4 years, or about 5 years, or about 6 years, or about 7 years, or about 8 years, or about 9 years, or about 10 years, including all values and ranges in between.

In embodiments, the treatment increases overall survival (OS) compared to either (i) the median OS of patients administered standard treatment for PTCL or (ii) the OS of an otherwise similar patient with PTCL that is not treated with duvelisib (e.g., a control patient). As used herein, OS is the length of time from diagnosis with PTCL until death. In embodiments, treating according to the methods of the disclosure results in an increase in OS as compared to the median value in overall survival at the time of this disclosure for peripheral T-cell lymphoma. In embodiments, treating according to the methods of the disclosure results in an increase in the OS of about 1 week, or about 2 weeks, or about 3 weeks, or about 4 weeks, or about 5 weeks, or about 6 weeks, or about 7 weeks, or about 8 weeks, or about 9 weeks, or about 10 weeks, or about 1 month, or about 2 months, or about 3 months, or about 4 months, or about 5 months, or about 6 months, or about 7 months, or about 8 months, or about 9 months, or about 10 months, or about 11 months, or about 12 months, or about 13 months, or about 14 months, or 15 months, or about 16 months, or about 17 months, or about 18 months, or about 19 months, or about 20 months, or about 21 months, or about 22 months, or about 23 months, or about 24 months, or about 3 years, or about 4 years, or about 5 years, or about 6 years, or about 7 years, or about 8 years, or about 9 years, or about 10 years, or more, including all values and ranges in between, compared to either (i) the median OS of patients administered standard treatment for PTCL or (ii) the OS of an otherwise similar patient with PTCL that is not treated with duvelisib. For example, in embodiments, the median overall survival for PTCL patients administered standard treatment is less than 6 months.

In embodiments, treating according to the methods of the disclosure results in an increase in the OS of about 10%, about 20%, about 30%, about 40%, about 50%, about 60%, about 70%, about 80%, about 90%, about 100%, about 150%, about 200%, about 250%, about 300%, about 350%, about 400%, about 450%, about 500%, about 550%, about 600%, about 650%, about 700%, about 750%, about 800%, about 850%, about 900%, about 950%, or about 1000% or more, including all values and ranges in between.

In embodiments, treating according to the methods of the disclosure results in OS of at more than 6 months, e.g., at least about 7 months, at least about 8 months, at least about 9 months, at least about 10 months, at least about 11 months, at least about 12 months, at least about 13 months, at least about 14 months, at least 15 months, at least about 16 months, at least about 17 months, at least about 18 months, at least about 19 months, at least about 20 months, at least about 21 months, at least about 22 months, at least about 23 months, at least about 24 months, at least about 3 years, at least about 4 years, at least about 5 years, at least about 6 years, at least about 7 years, at least about 8 years, at least about 9 years, at least about 10 years, or more, including all values and ranges in between.

In embodiments, the treatment increases duration of response (DOR) compared to either (i) the median DOR of patients administered standard treatment for peripheral T-cell lymphoma or (ii) the DOR of an otherwise similar patient with peripheral T-cell lymphoma that is not treated with duvelisib (e.g., a control patient). As defined herein, DOR is the length of time from a response to treatment until death. In embodiments, treating according to the methods of the disclosure results in an increase in DOR as compared to the median value in DOR at the time of this disclosure for peripheral T-cell lymphoma. For example, the median DOR for peripheral T-cell lymphoma patients whose cancer has metastasized less than 6 months. In embodiments, treating according to the methods of the disclosure results in an increase in the DOR of about 1 week, or about 2 weeks, or about 3 weeks, or about 4 weeks, or about 5 weeks, or about 6 weeks, or about 7 weeks, or about 8 weeks, or about 9 weeks, or about 10 weeks, or about 1 month, or about 2 months, or about 3 months, or about 4 months, or about 5 months, or about 6 months, or about 7 months, or about 8 months, or about 9 months, or about 10 months, or about 11 months, or about 12 months, or about 13 months, or about 14 months, or 15 months, or about 16 months, or about 17 months, or about 18 months, or about 19 months, or about 20 months, or about 21 months, or about 22 months, or about 23 months, or about 24 months, or about 3 years, or about 4 years, or about 5 years, or about 6 years, or about 7 years, or about 8 years, or about 9 years, or about 10 years, or more, including all values and ranges in between.

In embodiments, treating according to the methods of the disclosure results in an increase in the DOR of about 10%, about 20%, about 30%, about 40%, about 50%, about 60%, about 70%, about 80%, about 90%, about 100%, about 150%, about 200%, about 250%, about 300%, about 350%, about 400%, about 450%, about 500%, about 550%, about 600%, about 650%, about 700%, about 750%, about 800%, about 850%, about 900%, about 950%, or about 1000% or more, including all values and ranges in between, compared to either (i) the median DOR of patients administered standard treatment for peripheral T-cell lymphoma or (ii) the DOR of an otherwise similar patient with peripheral T-cell lymphoma that is not treated with duvelisib.

In embodiments, treating according to the methods of the disclosure results in DOR of at least about 6 months, at least about 7 months, at least about 8 months, at least about 9 months, at least about 10 months, at least about 11 months, at least about 12 months, at least about 13 months, at least about 14 months, at least 15 months, at least about 16 months, at least about 17 months, at least about 18 months, at least about 19 months, at least about 20 months, at least about 21 months, at least about 22 months, at least about 23 months, at least about 24 months, at least about 3 years, at least about 4 years, at least about 5 years, at least about 6 years, at least about 7 years, at least about 8 years, at least about 9 years, at least about 10 years, or more, including all values and ranges in between.

In embodiments, the treatment increases the disease control rate (DCR) compared to either (i) the median DCR of patients administered standard treatment for PTCL or (ii) the DCR of an otherwise similar patient with PTCL that is not treated with duvelisib (e.g., a control patient). As used herein, DCR is the proportion of patients in whom the best overall response is determined as complete response (CR), partial response (PR) or stable disease (SD). In embodiments, treating according to the methods of the disclosure results in an increase in DCR as compared to the median value in DCR at the time of this disclosure PTCL.

In embodiments, treating according to the methods of the disclosure results in an increase in the DCR of about 10%, about 20%, about 30%, about 40%, about 50%, about 60%, about 70%, about 80%, about 90%, about 100%, about 150%, about 200%, about 250%, about 300%, about 350%, about 400%, about 450%, about 500%, about 550%, about 600%, about 650%, about 700%, about 750%, about 800%, about 850%, about 900%, about 950%, or about 1000% or more, including all values and ranges in between, compared to either (i) the median DCR of patients administered standard treatment for PTCL or (ii) the DCR of an otherwise similar patient with PTCL that is not treated with duvelisib.

### Combinations

In embodiments, the method of treating PTCL comprises administration of duvelisib, and one or more other therapeutic agents. In embodiments, the other therapeutic agent is selected from a list consisting of 5-azacitidine, 6,8-bis(benzylthio)octanoic acid, 7-hydroxystaurosporine, ABT-263, adcetris, adriacin, adriamycin, AFM13, AK104, alefacept, alemtuzumab, alisertib, ALRN-6924, anti-CD30 CAR T cells, anti-CD7 CAR-pNK cells, anti-endosialin, anti-TRBC1 CAR-T cell therapy, apatinib, AR-42, araC, ASN002, ASTX660, ATLCAR.CD30 T cells, ATL-RIC, AUTO4, avelumab, azacidine, azacitidine, AZD4205, belinostat, bendamustine, bevacizumab, bleomycin, bortezomib, BR101801, brentuximab vedotin, busulfan, cabiralizumab, calaspargase pegol, camptothecin, carboplatin, carfilzomib, carmustine, cediranib, cerdulatinib, chidamide, cholecalciferol, cisplatin, cladribine, clin B, clofarabine, CT125A cells, cyclophosphamide, cyclosporine, cyclosporine A, cytarabine, dacarbazine, daratumumab, darinaparsin, dasatinib, decitabine, deferasirox, denileukin diftitox, deoxycoformycin, depsipeptide, dexamethasone, dexrazoxane, doxil, doxorubicin, DS-3201b, durvalumab, E7777, Endostar, endoxan, epirubicin, epratuzumab, etoposide, everolimus, F520, fenretinide, filgrastim, fludarabine, folic acid, folotyn, forodesine, GB226, gemcitabine, golimumab, granulocyte-colony stimulating factor, GVHD prophylaxis, HBI-8000, HDAC inhibitor, hydrocortisone, hydroxydaunorubicin, ifosfamide, imetelstat, infliximab, ipilimumab, istodax, ixazomib, KPT-330, lacutamab, L-Asparaginase, LB1901, L-Bcl-2 antisense oligonucleotide, lenalidomide, letermovir, leucovorin, loratadine, MEDI-570, melphalan, methotrexate, methoxyamine, methylprednisolone, mitoxantrone, MK2206, MK-3475, MLN9708, mogamulizumab, MORAb-004, MRIC, mycophenolate mofetil, navelbine, nelarabine, nelfinavir, nivolumab, 06-benzylguanine, obatoclax, OT-82, oxaliplatin, oxaliplatine, paclitaxel, panobinostat, PD-1 antibody, pegfilgrastim, pegylated liposomal doxorubicin, pembrolizumab, pentostatin, pharmorubicin, plerixafor, PLM60, pralatraxate, pralatrexate, prednisolone, prednisone, r-(−)-gossypol acetic acid, RAD001, rhIL-15, RIC, rituximab, romidepsin, ruxolitinib, sasanlimab, SEA-TGT, selinexor, SHC014748M, sintilimab, siplizumab, sorafenib, STI-3031, sunitinib, tacrolimus, TAK228, TAS4464, temsirolimus, TGR-1202, thiotepa, thymoglobulin, tipifarnib, tislelizumab, TQ-B3525, TRU-016, TTI-621, tucidinostat, valemetostat, valproic acid, velcade, venetoclax, vincristine, vindesine, VIP/ABVD, volasertib, vorinostat, Y 90 anti-CD45 monoclonal antibody BC8, Y 90 Basiliximab, YY-20394 and combinations thereof.

### Pharmaceutical Formulations

The therapeutic agents described here may be formulated in any suitable pharmaceutical composition. In embodiments, provided herein is a pharmaceutical composition for oral administration, wherein the composition comprises pharmaceutically acceptable salts, hydrates, solvates thereof, and a pharmaceutically acceptable excipient (e.g., an excipient suitable for oral administration).

In some embodiments, the composition provided herein comprises a polymorph of duvelisib. For example, polymorphs as described in WO 2012/097000, which are incorporated herein by reference for all purposes. In some embodiments, the composition provided herein comprises a mixture of two or more polymorphs of duvelisib, or a pharmaceutically acceptable salt, solvate, or hydrate thereof, e.g., polymorphs described herein.

In some embodiments, provided herein is a solid pharmaceutical composition suitable for oral administration, comprising: a therapeutically effective amount of a PI3K inhibitor (e.g., duvelisib) disclosed herein or a pharmaceutically acceptable form (e.g., pharmaceutically acceptable salts, hydrates, and solvates) thereof, and one or more pharmaceutical excipients suitable for oral administration.

In certain embodiments, pharmaceutical compositions provided herein suitable for oral administration can be presented as discrete dosage forms, such as capsules, pills, cachets, or tablets. In embodiments, the composition provided herein is a tablet or a capsule. In general, for solid forms, the compositions are prepared by uniformly and intimately admixing the active ingredient with liquid carriers or finely divided solid carriers or both, and then, if necessary, shaping the product into a certain presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid or semi-solid diluent.

In embodiments, the active ingredient can optionally be mixed with one or more inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) disintegrating agents such as agar, calcium carbonate, potato or tapioca starch, alginic acid, certain silicates, and sodium carbonate, and d) lubricants such as talc, calcium stearate, magnesium stearate, solid polyethylene glycols, sodium lauryl sulfate, and mixtures thereof. In the case of capsules, tablets and pills, the dosage form can comprise buffering agents.

In certain embodiments, binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures of two or more thereof. In some embodiments, exemplary binding agents include, but are not limited to, starch (e.g. cornstarch and starch paste); gelatin; sugars (e.g. sucrose, glucose, dextrose, dextrin, molasses, lactose, lactitol, mannitol, etc.); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan); alginates; polyethylene oxide; polyethylene glycol; inorganic calcium salts; silicic acid; polymethacrylates; waxes; water; alcohol; etc.; and mixtures of two or more thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures of two or more thereof.

In certain embodiments, disintegrants can be used in the compositions provided herein to provide tablets that disintegrate when exposed to an aqueous environment. The amount of disintegrant used can vary based upon the type of formulation and mode of administration. In certain embodiments, about 0.5 to about 15 weight percent of disintegrant, or about 1 to about 5 weight percent of disintegrant, can be used in a pharmaceutical composition provided herein. Disintegrants that can be used to form pharmaceutical compositions and dosage forms provided herein include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures of two or more thereof.

In certain embodiments, lubricants which can be used to form pharmaceutical compositions and dosage forms provided herein include, but are not limited to, calcium stearate, magnesium stearate, mineral oil, light mineral oil, glycerin, glyceryl behanate, sorbitol, mannitol, polyethylene glycol, other glycols, stearic acid, sodium lauryl sulfate, sodium benzoate, sodium acetate, sodium chloride, leucine, magnesium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, malt, and mixtures of two or more thereof. Additional lubricants include, for example, a syloid silica gel, a coagulated aerosol of synthetic silica, or mixtures of two or more thereof. In certain embodiments, a lubricant can optionally be added, in an amount of less than about 1 weight percent of the pharmaceutical composition.

In certain embodiments, tablets can be uncoated or coated by known techniques to delay disintegration and absorption in the gastrointestinal tract and thereby provide a sustained action over a longer period. For example, a time delay material, such as glyceryl monostearate or glyceryl distearate, can be employed. Formulations for oral use can also be presented as hard gelatin capsules, wherein the active ingredient is mixed with an inert solid diluent, for example, calcium carbonate, calcium phosphate, or kaolin; or as soft gelatin capsules, wherein the active ingredient is mixed with water or an oil medium, for example, peanut oil, liquid paraffin, or olive oil.

In another embodiment, an acid or a base can be incorporated into a composition provided herein to facilitate processing, to enhance stability, or for other reasons. Examples of pharmaceutically acceptable bases include, but are not limited to, amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS), and the like. In certain embodiments, pharmaceutically acceptable bases are salts of a pharmaceutically acceptable acid. Examples of pharmaceutically acceptable acids include, but are not limited to, acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like; and salts of polyprotic acids, such as sodium phosphate, disodium hydrogen phosphate, and sodium dihydrogen phosphate. When the base is a salt, the cation can be any convenient and pharmaceutically acceptable cation, such as ammonium, alkali metals, alkaline earth metals, and the like. Example can include, but not limited to, sodium, potassium, lithium, magnesium, calcium and ammonium.

In embodiments, the composition contains colloidal silicon dioxide, crospovidone, magnesium stearate, and microcrystalline cellulose. In embodiments, the capsule shell contains gelatin, titanium dioxide, black ink, and red iron oxide.

### Numbered Embodiments

Embodiment 1. A method of treating peripheral T-cell lymphoma in a subject in need thereof, comprising administering to the subject a therapeutically effective amount of a PI3K inhibitor in three or more treatment cycles; wherein a total daily dose of the PI3K inhibitor in the first two treatment cycles is higher than a total daily dose of the PI3K inhibitor in one or more subsequent treatment cycles.

Embodiment 2. The method of embodiment 1, wherein the PI3K inhibitor is selected from the list consisting of duvelisib, idelalisib, copanlisib, parsaclisib, paxalisib, umbralisib, and zandelisib.

Embodiment 3. The method of embodiment 2, wherein the PI3K inhibitor is duvelisib or a pharmaceutically acceptable salt or hydrate thereof.

Embodiment 4. The method of embodiment 3, wherein the duvelisib is a hydrate.

Embodiment 5. The method of embodiments 1-4, wherein each treatment cycle has a duration of about 28 days.

Embodiment 6. The method of embodiments 1-5, wherein total daily dose of the PI3K inhibitor of the first two treatment cycles is about 100 mg to about 200 mg.

Embodiment 7. The method of embodiment 6, wherein total daily dose of the PI3K inhibitor of the first two treatment cycles is about 150 mg.

Embodiment 8. The method of embodiments 1-5, wherein about 75 mg of the PI3K inhibitor is administered twice daily during the first two treatment cycles.

Embodiment 9. The method of embodiments 1-7, wherein total daily dose of the PI3K inhibitor in one or more subsequent treatment cycles is about 40 mg to about 60 mg.

Embodiment 10. The method of embodiment 8, wherein total daily dose of the PI3K inhibitor in one or more subsequent treatment cycles is about 50 mg.

Embodiment 11. The method of embodiments 1-5, wherein about 25 mg of the PI3K inhibitor is administered twice daily during the third treatment cycle.

Embodiment 12. The method of embodiments 1-7 and 8-10, wherein the PI3K inhibitor is administered twice daily.

Embodiment 13. The method of embodiment 12, wherein the peripheral T-cell lymphoma is peripheral T-cell lymphoma not other specified (PTCL-NOS), anaplastic large cell lymphoma (ALCL), natural-killer/T-cell lymphoma (NKTL), or angioimmunoblastic T-cell lymphoma (AITL).

Embodiment 14. The method of embodiment 13, wherein the peripheral T-cell lymphoma is PTCL-NOS.

Embodiment 15. The method of embodiment 13, wherein the peripheral T-cell lymphoma is ALCL.

Embodiment 16. The method of embodiment 13, wherein the peripheral T-cell lymphoma is AITL.

Embodiment 17. The method of embodiment 13, wherein the peripheral T-cell lymphoma is NKTL.

Embodiment 18. The method of embodiments 1-17, wherein the lymphoma is relapsed or refractory.

Embodiment 19. The method of embodiment 18, wherein the subject received at least 2 cycles of one prior regimen and one of the following:
a. failed to achieve at least a partial response after 2 or more cycles;
b. failed to achieve a complete response after 6 or more cycles; and/or
c. progressed after an initial response.

Embodiment 20. The method of embodiment 1-19, wherein the subject received prophylaxis treatment to pneumocystis jirovecii pneumonia (PJP), herpes simplex virus (HSV), varicella zoster virus (VZV) prophylaxis, or combinations thereof.

### EXAMPLES

### Example 1: Clinical Trial of Duvelisib in Patients with Relapsed or Refractory Peripheral T-Cell Lymphoma (R/R PTCL)—Trial 1

A phase 2, open-label, multi-center, parallel cohort trial of duvelisib in R/R PTCL was carried out. Patients were treated with duvelisib for two 28-day cycles with either 25 mg BID (DUV25) or 75 mg BID (DUV75). Patients with pathologically confirmed R/R PTCL (WHO criteria) after ≥2 cycles of ≥1 prior standard regimen, and a CD4 lymphocyte count of ≥50/mm³ were eligible for enrollment in the study. The initial results of the dose-optimization phase (N=33) demonstrated a 54% ORR in the 75 mg BID (N=13) and 35% in the 25 mg BID (N=20) cohorts. The following were identified as treatment emergent adverse events:
Serious TEAEs in ≥2 patients were colitis, diarrhea, abdominal pain, pyrexia, PD, sepsis, pneumonia, hyponatremia, rash maculopapular, dyspnea, and respiratory failure
Serious TEAEs occurred in 75% of DUV25 (n=15/20) and 69% of DUV75 (n=9/13)
There were 5 fatal AEs in DUV25 (3 disease progression, 1 death, 1 respiratory disorder/sepsis, 1 cardiac arrest/respiratory failure) and 1 in DUV75 (disease progression)
Most TEAEs were manageable with dose reductions and dose holds
Discontinuations due to AEs were higher in DUV25 (30%) vs DUV75 (8%)
Some subjects had >1 AE leading to discontinuation
TEAEs leading to discontinuation in DUV25 were disease progression, pneumonitis (2 patients each), cardiac arrest, hypercalcemia, hyperglycemia, pneumonia, rash maculopapular, and respiratory failure (1 each)
TEAEs leading to discontinuation in DUV75 were disease progression, infectious colitis, and sepsis (1 each)

DUV75 resulted in less discontinuations from adverse events; however, the side effects for DUV75 included infectious colitis and sepsis, which was not present in DUV25.

### Example 2: Clinical Trial of Duvelisib in Patients with Relapsed or Refractory Peripheral T-Cell Lymphoma (RIR PTCL)—Trial 2

An expansion phase of the above trial described in Example 1 was carried out. Based on the dose optimization results, patients in the expansion phase received duvelisib at 75 mg BID for 2 cycles to maximize rapid tumor control, followed by 25 mg BID to maintain long-term disease control and mitigate late toxicities, until progressive disease (PD) or unacceptable toxicity. The primary endpoint was the overall response rates (ORR) as assessed by the Independent Review Committee (IRC), and secondary endpoints included ORR by INV assessment, duration of response (DOR), progression free survival (PFS), overall survival (OS), disease control rate (DCR), and safety. Patients had a median age of 67.0 years (range, 21-92 years) and a median of 3 prior lines of therapy (range, 1-9). Analysis was performed on data collected from a total of 101 patients with a median follow-up of 8.7 months from time of first response. All patients had pathologically confirmed PTCL (WHO criteria) with a CD4 lymphocyte count of ≥50/mm³ and received ≥2 cycles of ≥1 prior standard regimen. Prior treatments received included CHOP/R-CHOP, CHOEP/EPOCH, or BV/BV-chemo (all 36.60% r), salvage chemo after CHOP/R-CHOP or CHOEP/EPOCH (37.60), and SCT (21.80). The mean treatment duration was 17.1 weeks, and the median (range) treatment duration was 9.0 (1-79) weeks. Exposure by treatment duration included 43.6% (≤2 cycles), 23.8% (>2-4 cycles), and 30.700 (>4 cycles). The ORR by IRC was 4900 with a CR rate of 340%. In patients with ≥3 prior lines, the ORR was maintained (49.10%). BOR (Best Overall Response; Lugano classification) by prior regimens and prior anticancer therapy are shown in Table 1.

TABLE 1
Preliminary Outcomes by Prior Regimens
and Prior Anticancer Therapy

| OUTCOME | PRIMO-EP (N = 101) |
|---|---|
| ORR by IRC, n (%) [95% CI] | 49 (48.5) [38.8-58.3] |
| CR by IRC, n (%) [95% CI] | 34 (33.7) [24.4-42.9] |

Response (CR + PR) by number of prior regimens, %

| | |
|---|---|
| 1 prior regimen (n = 26) | 34.6 |
| 2 prior regimens (n = 22) | 63.6 |
| 3+ prior regimens (n = 53) | 49.1 |

BOR by prior therapy, (% of subgroup): CR/PR/SD

| | |
|---|---|
| Prior CHOP/R-CHOP | 37.8/5.4/2.7 |
| Prior CHOEP/EPOCH | 27.0/27.0/0 |
| Prior Salvage Chemotherapy CHOP/R-CHOP or | 44.7/7.9/0 |
| CHOEP/EPOCH | |
| Prior BV or BV-chemo | 32.4/10.8/0 |
| Prior SCT | 22.7/27.3/0 |

BV-chemo, brentuximab vedotin + cyclophosphamide + doxorubicin + prednisone; CHOEP, cyclophosphamide + doxorubicin + vincristine + etoposide + prednisone; CHOP, cyclophosphamide + doxorubicin + vincristine + prednisone; EPOCH, etoposide + prednisone + vincristine + cyclophosphamide + doxorubicin; R-CHOP, rituximab + CHOP.

Median (range) PFS was 3.62 (0.03+-17.2+) months. Median duration of response was 7.7 months, and 7.4 months for those in CR. TEAEs leading to dose hold or dose reduction occurred in 37.6% and 3.0% of patients. The most frequent adverse events (AEs) (all causality) with maximum GR3 were increased alanine aminotransferase/aspartate aminotransferase (ALT/AST) (14.9%/13.4%), rash maculo-papular and diarrhea (both 7.9%) neutropenia (13.8%) and increased ALT (5.9%, maximum GR4). Adverse events of special interest (AESI) included infections, colitis, cutaneous reactions, neutropenia, diarrhea, pneumonitis, and transaminase elevation. Incidence of transaminase elevation was less at Cycle >4 (12.9%), versus at Cycle ≤2 (33.7%). Incidence of pneumonia, pneumonitis, and colitis was low regardless of treatment duration (Cycle ≤2 to Cycle >4: range 0-3.2%), and the rates of some AESIs increased slightly from Cycle ≤2 to Cycle >4 (infections increased by 5.6%, diarrhea and cutaneous reactions both increased by 2.8%). Transaminase elevations decreased, and diarrhea and infections increased as number of prior anticancer regimens increased.

Total 8 deaths in TEAEs other than PD were observed as follows: 1 patient each experiencing GI hemorrhage, vascular dementia, acute cholecystitis, hypoxia, suicide (unlikely/unrelated), and sepsis, EBV-associated lymphoproliferative disorder, and pneumonitis (treatment-related). AESIs by select subgroups: See Table 2.

TABLE 2
Preliminary Results: Adverse events of special interest (all grades,
all causality) by prior regimen and time on treatment subgroups

| | Adverse event of special interest, n (%) | | | | | | | | | | | |
|---|---|---|---|---|---|---|---|---|---|---|---|---|
| | | | | | | | Tx | | Tx | | Tx | |
| | 1 prior | | 2 prior | | 3+ prior | | Duration ≤2 | | Duration >2-4 | | Duration >4 | |
| | regimen | | regimens | | regimens | | cycles | | cycles | | cycles | |
| | (N = 26) | | (N = 22) | | (N = 53) | | (N = 101) | | (N = 56) | | (N = 31) | |
| Infections | 8 | (30.8) | 7 | (31.8) | 26 | (49.1) | 27 | (26.7) | 9 | (16.1) | 10 | (32.3) |
| Colitis | 0 | | 0 | | 1 | (1.9) | 0 | | 0 | | 1 | (3.2) |
| Cutaneous | 10 | (38.5) | 12 | (54.5) | 13 | (24.5) | 20 | (19.8) | 16 | (28.6) | 7 | (22.6) |
| reactions | | | | | | | | | | | | |
| Diarrhea | 4 | (15.4) | 6 | (27.3) | 21 | (39.6) | 20 | (19.8) | 8 | (14.3) | 7 | (22.6) |
| Neutropenia/ | 10 | (38.5) | 6 | (27.3) | 17 | (32.1) | 29 | (28.7) | 8 | (14.3) | 8 | (25.8) |
| neutrophil count | | | | | | | | | | | | |
| decreased | | | | | | | | | | | | |
| Pneumonia | 1 | (3.8) | 0 | | 2 | (3.8) | 2 | (2.0) | 0 | | 1 | (3.2) |
| Pneumonitis | 1 | (3.8) | 0 | | 1 | (1.9) | 1 | (1.0) | 1 | (1.8) | 0 | |
| Transaminase | 17 | (65.4) | 9 | (40.9) | 18 | (34.0) | 34 | (33.7) | 17 | (30.4) | 4 | (12.9) |
| elevation | | | | | | | | | | | | |

The interim results of the expansion phase show an ORR of 49% and a CR rate of 34%, which suggests this therapy is superior to currently available standard of care (SOC) therapeutic options. Duvelisib was well tolerated in this population and remained consistent with the known safety profile of duvelisib. These data, from a large diverse population of T-cell lymphoma patients, build upon prior reports demonstrating duvelisib as an active oral treatment for patients with R/R PTCL.

### CONCLUSION

The response by type of prior anticancer therapy was generally consistent with the overall population (range 43.2% to 54%). The response rates of ≥35% were seen regardless of number of prior regimens. The types of AEs seen were consistent with those observed previously in the PRIMO trial. As shown in Table 2, the rates of AESIs (infections, cutaneous reactions, diarrhea, decreased neutropenia/neutrophil count, pneumonia, pneumonitis, and transaminase elevation) significantly decreased in patients who were treated with the >2-4 and >4 cycles treatment regimens as compared to the patients who received ≤2 cycle treatment.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

What is claimed is:

1. A method of treating peripheral T-cell lymphoma in a subject in need thereof, comprising orally administering to the subject for three or more treatment cycles duvelisib having the following structure: 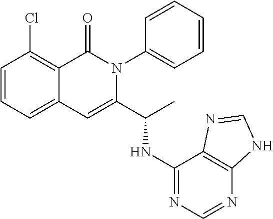 [SMILES: C[C@H](Nc1ncnc2[nH]cnc12)c1cc2cccc(Cl)c2c(=O)n1-c1ccccc1]
or a pharmaceutically acceptable salt or hydrate thereof;
wherein about 75 mg of duvelisib is administered twice daily for first two treatment cycles; and
wherein about 25 mg of duvelisib is administered twice daily for at least the third treatment cycle.

2. The method of claim 1, wherein duvelisib is a hydrate.

3. The method of claim 1, wherein each treatment cycle has a duration of about 28 days.

4. The method of claim 1, wherein 25 mg of duvelisib is administered twice daily in one or more treatment cycles following the third treatment cycle.

5. The method of claim 1, wherein 25 mg of duvelisib is administered twice daily in each treatment cycle following the third treatment cycle.

6. The method of claim 1, comprising at least a fourth, fifth, six, seventh, eighth, ninth, tenth, eleventh, and twelfth treatment cycles, and the method comprises administering 25 mg of duvelisib twice daily in one or more of the fourth, fifth, six, seventh, eighth, ninth, tenth, eleventh, and twelfth treatment cycles.

7. The method of claim 1, comprising at least a fourth, fifth, six, seventh, eighth, ninth, tenth, eleventh, and twelfth treatment cycles, and the method comprises administering 25 mg of duvelisib twice daily in two, three, four, five, six, seven, eight, or ten of the fourth, fifth, six, seventh, eighth, ninth, tenth, eleventh, and twelfth treatment cycles.

8. The method of claim 1, wherein the peripheral T-cell lymphoma is peripheral T-cell lymphoma not other specified (PTCL-NOS), anaplastic large cell lymphoma (ALCL), natural-killer/T-cell lymphoma (NKTL), or angioimmunoblastic T-cell lymphoma (AITL).

9. The method of claim 8, wherein the peripheral T-cell lymphoma is PTCL-NOS.

10. The method of claim 8, wherein the peripheral T-cell lymphoma is ALCL.

11. The method of claim 8, wherein the peripheral T-cell lymphoma is AITL.

12. The method of claim 8, wherein the peripheral T-cell lymphoma is NKTL.

13. The method of claim 1-12, wherein the PTCL is relapsed or refractory.

14. The method of claim 13, wherein the subject received at least 2 cycles of one prior treatment regimen for PTCL and:
a. failed to achieve at least a partial response after 2 or more cycles;
b. failed to achieve a complete response after 6 or more cycles; and/or
c. progressed after an initial response.

15. The method of claim 1-19, comprising administering to the subject prophylaxis treatment for pneumocystis jirovecii pneumonia (PJP), herpes simplex virus (HSV), varicella zoster virus (VZV) prophylaxis, or combinations thereof.

16. The method of any one of the preceding claims, wherein treatment increases the overall response rates (ORR) compared to either (i) the median ORR of patients administered standard treatment for PTCL or (ii) the ORR of an otherwise similar patient with PTCL that is not treated with duvelisib.

17. The method of any one of the preceding claims, wherein treatment increases progression free survival (PFS) compared to either (i) the median PFS of patients administered standard treatment for PTCL or (ii) the PFS of an otherwise similar patient with PTCL that is not treated with duvelisib.

18. The method of any one of the preceding claims, wherein treatment increases overall survival (OS) compared to either (i) the median OS of patients administered standard treatment for PTCL or (ii) the OS of an otherwise similar patient with PTCL that is not treated with duvelisib.

19. The method of any one of the preceding claims, wherein treatment increases duration of response (DOR) to either (i) the median DOR of patients administered standard treatment for PTCL or (ii) the DOR of an otherwise similar patient with PTCL that is not treated with duvelisib.

20. The method of any one of the preceding claims, wherein treatment increases the disease control rate (DCR) to either (i) the median DCR of patients administered standard treatment for PTCL or (ii) the DCR of an otherwise similar patient with PTCL that is not treated with duvelisib.

21. The method of any one of the preceding claims, wherein the administration provides overall response rates (ORR) of about 30% to about 60%.

22. The method of any one of the preceding claims, wherein the administration provides overall response rates (ORR) of about 49%.

23. The method of any one of the preceding claims, wherein the administration provides progression free survival (PFS) of about 3 months to about 21 months.

24. The method of any one of the preceding claims, wherein the administration provides progression free survival (PFS) of about 3.6 months.

25. The method of any one of the preceding claims, wherein the administration provides overall survival (OS) of about 6 months or longer.

26. The method of any one of the preceding claims, wherein the administration provides duration of response (DOR) of about 7 months to about 8 months.
